(19) United States
(12) Patent Application Publication
(10) Pub. No.: US 2002/0123144 A1
(43) Pub. Date: Sep. 5, 2002

(54) Method for producing synchronous cells and the resultant cells

(76) Inventor: Charles Helmstetter, Satellite Beach, FL (US)

Correspondence Address: POLSINELLI SHALTON & WELTE, P.C., 700 W. 47TH STREET, SUITE 1000, KANSAS CITY, MO, 64112-1802, US

(21) Appl. No.: 10/080,333

(22) Filed: Feb. 21, 2002

Related U.S. Application Data
(60) Provisional application No. 60/273,775, filed on Mar. 2, 2001.

(51) Int. Cl. C12N 5/08; C12N 5/06

(52) U.S. Cl. 435/372; 435/325

(57) ABSTRACT

The present invention relates to a method for producing a synchronous cell culture and the resulting cells, whereby the cells are of the same size and the same mitotic age. In particular, the present cells are mammalian in nature and, preferably hematopoietic cells.

The present application is based on Provisional Patent Application Serial No. 60/273,775, which was filed on Mar. 2, 2001.

The U.S. Government has a paid-up license in this invention and the right in limited circumstances to require the patent owner to license others on reasonable terms as provided for by the terms of National Institutes of Health Award Nos. GM26429 and AG15187, and NASA Award Nos. NAG5-3886 and NAGW-4503.

### FIELD OF INVENTION

The present invention relates to a method for producing synchronous cell suspensions, which are, preferably, mammalian cell suspensions. Additionally, the present invention relates to the resultant synchronous cells, in particular, hematopoietic cells.

### BACKGROUND OF INVENTION

Thousands of biological research and development facilities worldwide deal with some aspect of the growth and division of cells. Suspensions of minimally disturbed newborn cells are extremely valuable for analysis of aspects of the cell cycle, including analysis of all mammalian cell types. The newborn cell suspensions can be used to analyze cell cycle gene expression in hematopoietic cells. Growing and dividing cells are used in basic research, drug development and testing, and bio-product manufacturing, for example. Furthermore, during the past few years there has been a significant expansion in research in the related areas of molecular biology of cell cycle regulation, cell senescence, apoptosis, and cellular differentiation. Such studies require access to, and reliable production of, large quantities of cells that are at a specific, known phase of growth and division, such as a particular stage in the cell cycle, and state of senescence. Obtaining adequate quantities of the appropriate cells, without disturbing their physiology, is not any easy task. For these reasons, it is desired to have a method for producing large quantities of cells at a known phase of growth and division.

Two basic types of methods are known and have been attempted for production of cells at specific stages of the cell cycle. The two known methods are referred to as induction and selection. Induction techniques involve treating a growing population in such a fashion as to convert a “randomly” growing culture into a population in which growth and division are “synchronized.” To be “synchronized” means a population of cells is at the same stage of growth and cell division. Synchronizing a culture can be achieved by starvation for serum or a required nutrient; temperature shifts; periodic changes in nutrition, heat, or light; exposure to excess thymidine; addition of inhibitors of specific macromolecular syntheses; and, combinations thereof. Many of these techniques are still in routine use, but they do not yield cells in an undisturbed steady-state cycle since the totality of cell processes is not aligned. Typically, the DNA molecules are not at the same stage of development. Further, any aligned growth that is achieved is likely compromised by the treatment procedure. The potential response of the cell property under investigation may vary with each population. Use of this method is disadvantageous because the cells have been damaged and disturbed.

To overcome problems associated with induction procedures, methods have been developed for selection of cells at specific stages in the division cycle. The known and preferred selection procedures involve obtaining or identifying cells of a fixed size or stage from a growing population by centrifugal elutriation, or flow cytometry/cell sorting to examine the properties of individual cells. Elutriation techniques have been used successfully with both prokaryotes and eukaryotes, although problems associated with disturbances caused by centrifugation for several minutes can limit the utility of this procedure. Mitotic selection has proven to be very successful for mammalian cells which grow adhered to surfaces and become less firmly adhered during mitosis but yields are limited unless inhibitors are added to accumulate cells in mitosis, and even this method can disturb cell growth. Flow cytometry has a number of advantages, including capability for cell cycle analyses in a wide range of cell types, and the ability to examine cells derived from tissues. When used as a cell sorter, however, it can be difficult to obtain sufficient quantities of cells at different stages in the cycle, and the precision of selection of cells at specific cycle stages is low. As can be seen, selection suffers from a number of problems.

It has been known to produce cell populations having synchronous growth that are not damaged. Previously, yeast cells and bacteria cultures could be treated to produce a synchronized population. In the known technology, bacteria are grown on a surface such that when a cell divides, one daughter cell remains adhered to the surface while the other daughter is released. This concept is shown schematically in FIG. 1 for generalized cells. When the original cells are attached firmly, the only cells that can be released are newborn cells whose surface is not involved in the initial attachment. The newborn cells collected from the effluent grew synchronously. It was believed, however, that hematopoietic cells could not be synchronized using the known method because it was believed the cells could not be adequately attached and held to a surface. Further, it was thought that even if the cells could be held, the plane of division would be such that a population could not be sustained which released newborn daughter cells. Hematopoietic cells do not have rigid walls, which led to the hypothesis that division would result in both cells sticking to the surface.

What is desired is to have a method that results in large quantities of pure cells that are at precisely known stages in the cell cycle and perhaps most importantly, minimally disturbed. Synchrony achieved by mitotic selection or elutriation would be expected to approach this critical objective, but even with these methods there can be disturbance of the cell cycle as judged by elongation of the first cycle after treatment. The availability of newborn cell suspensions would solve the problems faced by laboratories requiring cells, or cellular components, at specific, known stages during undisturbed growth and division.

This result cannot be achieved by any physical separation or chemical treatment. Current technologies that collect small cells by size cannot produce such populations, since not all newborn cells are small and not all small cells are newborn. Thus, it is desired to have large populations of synchronous cells. It is especially desired if this population is of a mammalian origin, in particular, hematopoietic cells.

### SUMMARY OF INVENTION

The present invention is related to method for producing a culture of cells that are synchronous and the resultant synchronous cells. In particular, the present invention relates to a method for producing a culture of newborn cells that are growing in a steady-state and have synchronous progression through the cell mitotic cycle, including cell cycle specific replication, transcription, and translation. Any of a variety of cells can be produced according to the present method, however, it is preferred if the cells are mammalian in origin and, more preferably, hematopoietic cells. As such, a culture of cells can be produced, whereby the culture has a population of at least 10³ newborn cells per milliliter (ml), with all the cells being roughly of the same mitotic age. The cells will have a similar size and have the same mitotic progression.

As mentioned, the present invention also relates to a method for producing the synchronous cell culture. The method includes contacting exponentially growing mammalian cells with a substrate member and adhering such cells to the substrate. The substrate can be any of a variety of materials on which cells can be adhered and which does not interfere with cell growth. Once attached, the cells will be continuously flushed with media, whereby the newborn cells will be washed away in the effluent and collected. The effluent will contain the newborn cell population.

The present invention also relates to a culture of parent cells, which produce the newborn daughter cells. The culture of parent cells should be an immortal cell line, or growth-activated normal cells, that are mammalian in origin. Obviously, it is preferred if the parent cells are hematopoietic cells.

The present invention is advantageous because a cell culture is produced that can be used in any of a variety of different research areas. The uniform nature of the cell culture is particularly advantageous because this allows for the study of cell aging characteristics, as well as analysis of DNA replication and progression. The present invention is particularly advantageous because the cell population is produced that is mitotically the same. Also, the cells are not damaged in any way.

### BRIEF DESCRIPTION OF DRAWINGS

FIG. 1 is a depiction of the scheme for growing cell cultures on adherent surfaces and the resultant release of daughter cells;

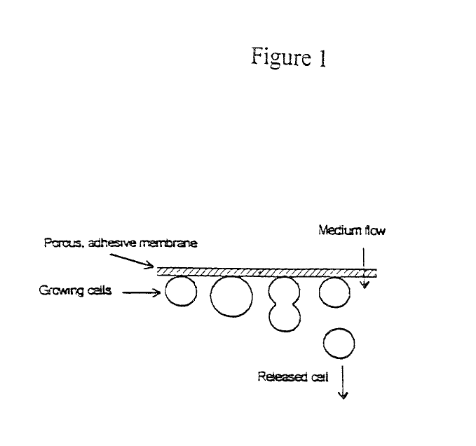

FIG. 2 is a schematic illustrating a generalized two step process for adhering cells to a surface;

FIG. 3 shows the theoretical and experimental concentration of newborn cells in the effluent from a membrane-bound population of L1210 cells;

FIG. 4 depicts cell size distributions of exponential-phase and baby machine-generated L1210 cells;

FIG. 5 shows synchronous growth of a sample collected between 3 and 3.5 hours (h) of elution of the culture shown in FIG. 3;

FIG. 6 depicts cell size distributions during synchronous growth of baby machine of L1210 cells generated with the present method;

FIG. 7 shows synchronous growth of newborn cell suspensions of L1210, MOLT-4, and U937 cells collected after approximately 0.3 generations of elution using the present method;

FIG. 8 depicts cell size distributions during synchronous growth of MOLT-4 and U937 cell suspensions;

FIG. 9 depicts DNA replication and cyclin A and B1 content during synchronous growth of MOLT-4 cell suspensions; and,

FIG. 10 depicts size distributions of newborn MOLT-4 and normal human lymphocyte suspensions generated with the present method.

### DETAILED DESCRIPTION

The present invention relates to a method for producing a synchronous mammalian cell population and the resulting population. More particularly, the present invention relates to a method for producing a synchronous population of hematopoietic cells. The resultant cells will typically be of the same size and be at the same stage of development, with the cells' DNA being essentially the same mitotically. The method relates to production of synchronous cell populations, in particular, cells that are of the same cell cycle, cell age, and differentiation. As such, the generation of synchronous newborn cell suspensions of hematopoietic cells, from both established cell lines and growth-activated normal cells, is accomplished. The suspensions so produced are minimally perturbed, thereby allowing investigations on cells in steady-state growth.

Newborn cell suspensions can be produced by the appropriate flushing of immobilized cell populations in a manner such that newborn cells, formed by cell divisions in the population, are withdrawn from the culture and collected. In this manner, newborn cell suspensions can be generated since by necessity all of the cells released from the immobilized population by cell division are newborn. To produce suspensions that are substantially comprised of newborn cells, the immobilized populations must be flushed uniformly and continuously with culture medium to withdraw the released cells from the surface and collect the suspensions of cells. The procedure for immobilization of the cells can vary widely but also depends on the type of newborn cell suspension required.

Any of a variety of eukaryotic and prokaryotic cells may be used with the present invention. It is preferred if the cells are of an animal origin, more preferably, mammalian in origin. The most preferred cells are differentiated stem cells, in particular, hematopoietic cells. Such cells can be derived from established cell lines or from growth activated normal cells. The cells must be such that they will undergo division during attachment. In order to form a sufficient population, it is preferred to start with a population equal to between 2×10⁵ and 3×10⁵ cells/ml. The number of cells added is dependent, in part, upon the cell size. Finally, the cells will typically be expanded to a sufficient population prior to attachment with the cells in a media solution prior to attachment.

One of the most preferred applications of the present culture system is to normal human lymphocytes. In addition to the use of newborn normal cells for cell cycle investigations, such cells can also be employed to investigate cell cycle changes as human cells age or senesce. It is well established that a variety of normal mammalian cells possess a limited replicative life span, e.g., fibroblasts and lymphocytes. In contrast, the “immortal” cell populations of the current invention can be maintained indefinitely. The present culture system is ideal for investigating changes in growth properties with replicative age since all aspects of the cell cycle can be measured automatically, as an entire culture ages. The attached cells will be an immortal cell line that produces multiple generations of newborn cells.

Once the population of cells is obtained, the cells are attached to a substrate member in any of a variety of ways, which include both affinity binding and adsorption. In affinity binding, a substrate surface is coated with a compound that will bind to receptors on the surface of the cell. The suitable compositions include charged molecules, antibodies, ligands (for which there are cell surface receptors), compounds that will form covalent bounds with surface molecules, lectins, agglutinins (which cause adherence), and combinations thereof. Specific examples of these compounds include lectins, antibodies to surface antigens, and fibronectin. In adsorption, the cells are bound to a surface coated with charged compounds such as, for example, a polyanion. The preferred coatings to be used with the differentiated stem cells include poly-D-lysine and concanavalin-A. However, any compound with affinity for the cells will do, including, but not limited to, charged molecules, antibodies, ligands, for which there are cell surface receptors, compounds that will form covalent bounds with surface molecules, lectins, and agglutinins.

Surprisingly, it would have been predicted by those familiar with the field, that if the surface was uniformly coated with adhesive, both newborn daughter cells would remain attached. However, it has been observed that hematopoietic cells have a high probability of dividing and separating away from the attachment substrate. It has been determined that division of the mammalian cells takes place in successive orthogonal plans. Resultingly, very few cell divisions occur along the plane of the substrate, so that one newborn cell is usually lifted from the surface.

As such, the coating composition will be added in an amount sufficient to hold a desired cell population to the substrate member. The coating is poured over the surface, preferably over a membrane, which allows passage of the media. The coating will adhere to the membrane. In the alternative, the coating can be spotted on a surface so that the cells attach to individual spots.

The manner of coating the membrane with adhesive does not appear to be important. Variations in concentrations of the polylysine, for example, vary within a 5-fold range, higher or lower than the preferred concentrations. Any amount of adhesive may be applied, as long as the cells can be held to the surface. The adhesive and the cells are preferably drawn onto the membrane slowly, about 1 ml/sec; however, this is not critical and both operations can be performed faster.

Any filter of similar pore size held in any of a variety of types of holders will perform as described. An example of a suitable filter is a 142 mm membrane filter, having a 0.22 μm pore size. As mentioned, a flat surface that is not porous can also be used in the alternative.

An alternative method for permanent attachment of cells to restricted, small sites is by a process involving physical, rather than chemical, attachment. In this procedure, cells are drawn to small holes in a non-adherent material through a differential pressure on the two sides of the material. The cells are held in place at the holes by the slight differential pressure, and when the cell divides, the daughter, having its surface at the hole, remains in place while the other daughter is released. In this embodiment, the immobilized cells “age” one generation each division cycle. The idea is that a cell is drawn to a hole in a solid surface, smaller than the cell diameter, and held in place with a slight pressure differential. When the cell divides, only the daughter associated with the hole remains attached and the other is released, since the pressure differential is blocked by the attached cell. It is thus a continuous culture device, producing newborn cells continuously, from a culture in steady-state growth. Thus, for normal cells, the cell products are not only at known cycle stages but known replicative ages for use in aging research.

For production of newborn cells during long term culture, and for examination of cellular aging in vitro, the attachment (chemical or physical) must be such that one sister cell remains attached, while one is released at most division. The number of attached cells in the culture unit does not change, or changes very little. This embodiment is applicable to most cell types, including adherent and non-adherent mammalian cells, bacteria, and yeast cells.

Once the cells are attached, the configuration of the attachment surfaces, and the flow of culture medium over the surfaces, must be optimized for collection of the newborn cells suspensions. Uniform flow of culture medium over the cells, at the appropriate rate, is required, whether the attachment surface consists of one or more flat plates or porous surfaces, or a column of adhesive elements. The flow can be across or over the cells. The flow must be such that newborn cells are washed away and collected as part of the effluent, without dislodging the attached cells. The media will be any of a variety of compositions necessary to establish and promote long term or short term growth and division in a cell population.

The resulting cells can have been cultured for an extended period of time in vitro. The parent cells can generate between 1 and 10 generations. The synchronous cultures are generated from cell lines established for in vitro growth, or from normal cells stimulated to grow in vitro through mitogenic stimulation. The synchronous cultures may be generated from mammals of varying ages or from cell cultures grown in vitro for varying time periods.

It is important to ensure that the cells on the surface are fully bathed in culture medium and that the released newborns are flushed from the surface. As stated, a variety of devices may be used, including devices designed to hold a filter while it is inverted, once the cells are attached. Any style filter holder can be used as long as the elution medium flows uniformly over the underside of the membrane when the holder is inverted. A funnel which will hold about 200 ml of culture medium above the membrane after it has been inverted works well since it introduces a slight sag in the membrane such that medium flow is uniform and single drops form which contain the daughter cells. A medium flow rate of 2 ml/min is preferred. A more preferred flow rate would be 0.002 ml/min/cm² or about 3 ml/day. Larger units, such as a unit having a surface area equal to 100 cm² can contain 5×10⁷ cells and will have a flow rate of 300 ml/day. The flow rates are dependent upon the structure, or device, which holds the cells. While the funnel and filter construction is preferred, any of a variety of devices may be used.

The resultant newborn cell cultures in effluent are comprised of synchronously growing cells, which may be used for the isolation and/or examination of the appearance, presence, and disappearance of individual cellular components during the mitotic cycle. The resulting cell population is minimally perturbed, of a uniform size, and of a synchronous progression through the mitotic cycle, including cycle-specific replication, transcription and translation, for more than one successive cycle. The culture will have at least 10³ cells/ml of effluent. The culture can have as little as 200 cells/minute. The present method can produce up to 10³ cells/min/cm². Stated another way, the present method can produce 5×10⁵ cells/generation/cm². Importantly, the resultant cell population will be at the same stage of cell division. As such, cultures can be produced that are comprised only of cells in either the S, G₁, G₂, or M, phases of the cell division cycle. The resultant suspension of newborn cells from growth-activated normal mammalian cells can be used for research on a normal cell cycle. Also, the initial cell population can be derived from mammals of varying ages to facilitate research on cell cycle aspects of cellular senescence. The cell suspension comprises cells cultured for extended periods in vitro for research on cell cycle aspects of cellular senescence, which is comprised of pluripotent hematopoietic stem cells. Thus, the resultant cell suspension can have been cultured for extended periods in vitro for research on cell cycle aspects of cellular senescence.

The following examples are for illustrative purposes only and are not to be construed as limiting the scope of the subject invention.

### EXAMPLES

### Example 1

The present example describes the production of suspensions of newborn mouse L1210 lymphocytic leukemia cells and their subsequent synchronous growth. As will be seen, the method produced minimally disturbed synchronous cells.

Mouse L1210 lymphocytic leukemia cells (ATCC CCL219) were cultured in spinner flasks in L-15 medium (Fischer) supplemented with 2 gm/l dextrose and 10% fetal bovine serum (GIBCO). The cells were grown for a sufficient period of time so that the experiments were initiated with exponentially growing cultures containing 2.6×10⁵ L1210 cells/ml in 300 ml of L-15 medium.

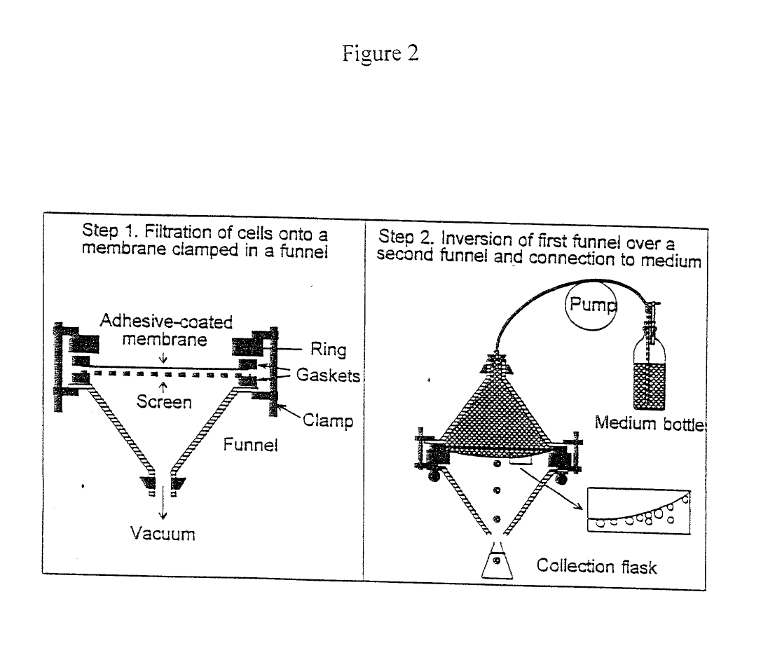

The apparatus used to produce the synchronous cell population consisted of a funnel designed to accommodate a 142 mm membrane filter. A cross sectional view of the membrane filter holder is displayed at FIG. 2, step 1. The lower section of the holder consisted of a 150 mm-diameter funnel machined from polymethylmethacrylate. A 142 mm diameter, 0.22 μm pore size nitrocellulose membrane filter (Millipore Corp, GSWP142) was supported by a stainless steel screen and placed on top of the lower funnel, with a plastic gasket between the screen and the funnel. A 20 mm thick polymethylmethacrylate ring (150 mm, OD) was placed above the nitrocellulose membrane, with a second gasket between the ring and the membrane. This entire holder was held together with aluminum clamps to form a tight seal.

Prior to use, the membrane filter was coated with poly-D-lysine. A solution consisting of 204 g/ml polylysine in 50 ml of phosphate buffered saline was poured onto the membrane and drawn through under vacuum at a rate of about 1 ml/second (sec). This resulted in a uniform coating of the membrane filter with poly-D-lysine, thereby forming a cell adherent substrate based on charge differences between the substrate and the cell surface. The membrane was then washed by drawing 100 ml of distilled water rapidly through the filter.

A culture of cells (250 ml) growing exponentially was poured onto the coated membrane, with the coated side facing up, and the medium was drawn through by vacuum at about 1 ml/sec. Filtration was stopped when a small amount of the culture remained above the membrane, to avoid drying the cells. The remaining fluid (about 10 ml) was poured off.

The filter holder was inverted and placed on top of a second plastic funnel resting on a ring stand. FIG. 2, step 2 shows the device during operation. The top of the holder was then filled with fresh culture medium (approximately 200 ml) and connected to a peristaltic pump. The filter holder was aligned so that the drops from the effluent passed freely through the hole in the second (lower) funnel. This arrangement maintained the temperature on the filter. The medium was pumped, from a 1 liter bottle containing a filtered vent, at a rate of 15 ml/min for the first 2 min to remove loosely attached cells and then reduced to 2 ml/min. The entire experiment was performed in a 37° C. incubator. The pump and culture media were held on a rack above the apparatus in the incubator. Air circulation within the incubator allowed a door to be opened for sample collection without altering the temperature within the chamber.

The resultant cells were collected and analyzed. The procedure as described produced approximately 5×10⁷ newborn cells per generation of elution with a surface area of approximately 100 cm². Cell concentrations ranged from 1 to 4×10⁵ cells per ml in 100 to 350 ml of total culture tested. Loading with about 5×10⁷ total cells appeared optimal, based on the purity of the newborn cell suspensions and their concentration.

As will be discussed, excellent synchrony was obtained by collecting samples for about 1/10 of the generation time, or about 1 h with these cells. Each synchronous sample contained, on average, about 5×10⁶ newborn cells. Since the daughter cells were shed continuously, sizable quantities of daughter cells for synchronous growth studies were obtained by collecting successive samples of the effluent and growing each for different times to obtain cells at different stages in the mitotic cycle.

### Example 2

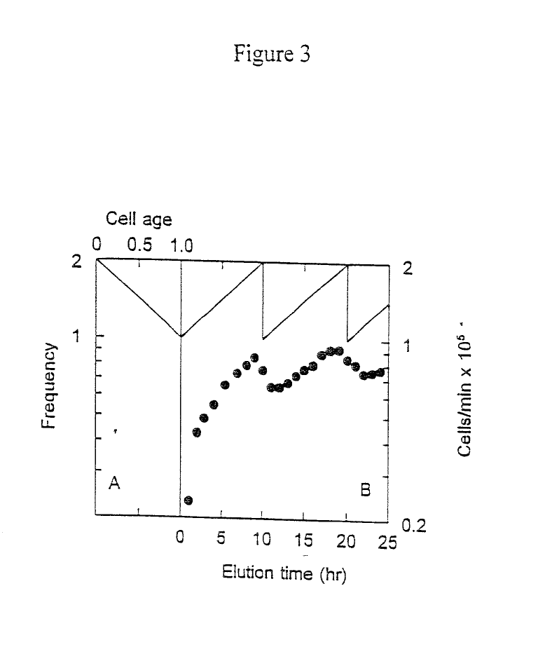

The resultant cells of Example 1 were analyzed. Cell concentrations were determined with a Coulter Electronic Particle Counter containing an aperture tube with a 100 μm diameter orifice. The data collected was used to develop FIG. 3.

FIG. 3 shows the concentration of cells shed into the effluent from the immobilized population. Line (A) is the idealized mitotic cycle age distribution in an exponential-phase culture containing no dispersion in generation times of individual cells. Line (B) shows the theoretical concentration of the cells in the effluent from a membrane-bound culture with the age distribution as shown in (A) and assuming one of the two newborn cells formed at division is shed from the surface. The experimental curve shows the number of cells in consecutive samples of the effluent from a membrane-bound culture of L1210 cells growing in L-15 medium at 37° C. A 234 ml culture containing 3×10⁵ cells/ml was filtered onto the surface. Four-minute samples were collected from the effluent starting at the indicated times. The shape of the curve is a function of the age distribution of the cells in the mitotic cycle in the exponentially growing population initially attached to the membrane surface. The theoretical idealized concentration of newborn cells in the effluent from the device is also shown, and is given by the mitotic cycle age distribution in reverse.

It was concluded that the first immobilized cells to divide and release one daughter (time 0) would be those that were closest to division at the time of attachment to the membrane. Thus, the relative number of newborn cells released into the effluent at the start of elution is given by the frequency of the oldest cells in the attached culture. As younger cells reach the division stage and release a daughter, the concentration of cells in the effluent increases reflecting the increasing frequency of their mothers in the original culture. At the end of the first generation of growth on the membrane, twice as many newborn cells were released because there were twice as many newborn cells as cells about to divide in the original exponential phase population. At this time, all of the original bound cells have divided once, and now begin dividing for the second time, again starting with the oldest cells. Thus, the theoretical cell concentration in the effluent produces a saw toothed shape curve varying two-fold in each generation.

The experimental results mimicked the theoretical curve except for the overall increase in the curve with time. This increase is likely a consequence of the frequency with which both daughters remain attached to the surface after division. The gradual rather than abrupt variations in the curve is a consequence of the dispersion in generation times of individual cells. The generation time of cells on the membrane was about 10 h, as given by the time between peaks, or the midpoints of the decreases in the elution curve. In this experiment, a total of approximately 5×10⁷ newborn cells were released in each generation. Since approximately 6.5×10⁷ cells were filtered onto the nitrocellulose membrane, the result indicated that about 90% of the cells attached to the surface.

### Example 3

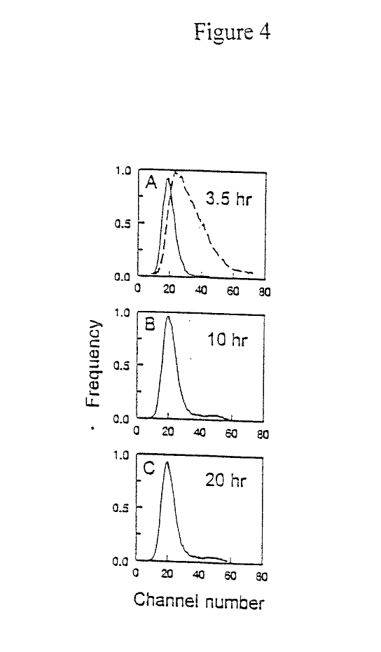

The cells of Example 1 were again analyzed. Evidence that the cells released from the immobilized population were virtually all newborn was based on size distributions, synchronous growth curves and cellular DNA distributions. Cell size distributions were determined with a Coulter Channelizer Model 256. The data was used to develop FIGS. 4a, 4b, and 4c.

FIG. 4a shows the size distribution of the cells in a sample of the effluent from the experiment discussed in Example 2, collected at 3.5 h, compared to the size distribution of cells in the exponential-phase culture prior to attachment. (A) Size distributions were determined with a Coulter electronic particle counter in a sample from an exponentially growing culture containing 3×10⁵ cells/ml ( . . . ), and in a sample collected after 3.5 h of elution of the same culture using the present method ( - - - ). FIGS. 4b and 4c demonstrate size distributions in samples collected after 10 h and 20 h of elution. The distribution of cell sizes in the effluent was narrow with a mean size corresponding to the very smallest cells in the exponential-phase culture.

The results demonstrate that essentially all of the cells shed from the membrane at 3.5 h of elution were newly formed daughters. FIGS. 4b and 4c show size distributions at the end of the first (10 h) and second (20 h) generations of elution. These size distributions remained essentially unchanged during the course of the experiment, except for the small number (about 5%) of larger cells. As shown, most of these larger cells are double the size of newborn, and appear microscopically as two newborn cells adhered together. The explanation for the doublets is unknown but they likely result from two released cells touching and adhering during division of adjacent immobilized cells or during passage along the membrane surface. In any event, the presence of cells other than newborn in the effluent is rare for at least 20 h. This demonstrates that the resultant cells were synchronized because the size of the cells remained virtually identical.

### Example 4

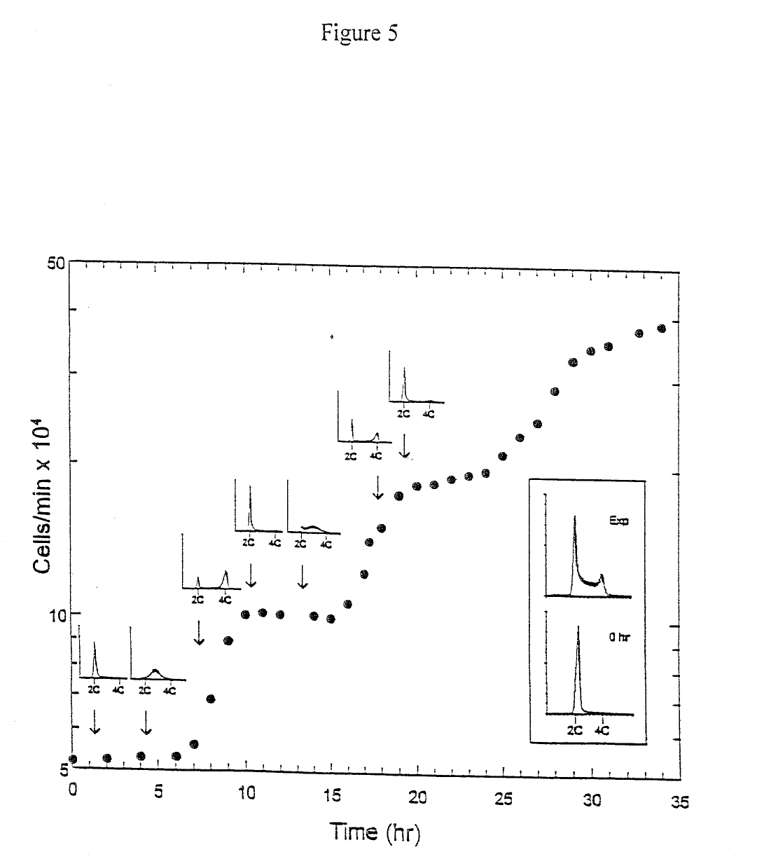

The DNA of the cells of Example 1 was analyzed. Cellular DNA distributions were determined with a FACScan flow cytometer (Becton Dickinson) after staining ethanol-fixed cells for 60 min in a solution of 504 g/ml of propidium iodide and 100 μg/ml of RNase A. The high quality and duration of synchronous growth of the newborn cells is evident in measurements of cell concentrations vs. time (FIG. 5), with the decay in synchrony being a natural consequence of the variation in interdivision times of individual cells.

As shown by FIG. 5, samples were taken from the synchronous culture in a 75 mm culture flask at the indicated times, beginning at the end of collection of the culture (time 0) for determination of cell concentrations. The DNA distributions determined by FACScan are also shown at the times indicated by the arrows during growth. The cells of different ages were obtained by collecting consecutive 10-min samples from the effluent starting at time 3.5 h and incubating each sample for different times in the cell cycle. The inset shows a comparison of the DNA distribution in an exponential-phase culture to that in a sample of the effluent. As expected for a highly synchronous culture, cells early in synchronous growth (1.5 h) contain G1 content of DNA (determined by flow cytometry), those about midway through the synchronous growth cycle (4.5 h) are in S, and those part way through synchronous division are in either G1 or G2 (7.5). This synchronous progression through DNA replication continued in the same manner through at least the start of the third cell cycle. The inset in the figure shows a comparison between the FACS DNA distributions in the exponential phase culture bound to the membrane and the newborn cells eluted at 3.5 h. Again, the synchronous nature of the cells was demonstrated.

### Example 5

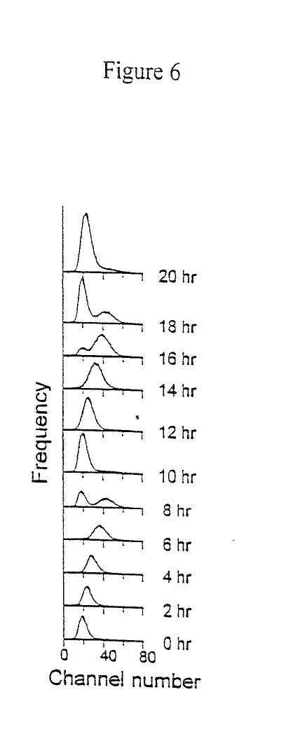

The cells of Example 1 were analyzed to determine size. The time course of changes in cell size distributions in the newborn cell cultures again demonstrated the high level of synchronous growth (FIG. 6). Size distributions were determined with a Coulter electronic particle counter in samples taken from the synchronous culture shown in FIG. 4 at the indicated times. The small cells at time 0 gradually increase in size uniformly during synchronous growth until they reach double the newborn cell size and then begin dividing at approximately 7 h. By 8 h, the cells are about midway through synchronous growth (FIG. 5) and show the clear bimodal size distribution corresponding to a mixture of newborn cells and cells just prior to division (FIG. 6). By 10 h, essentially all of the cells have divided to yield a single peak at approximately newborn size. This same pattern is clearly evident through the second synchronous growth cycle as well. Even at the start of the third cycle, the synchrony of the cells is apparent.

The above Examples (2-5) demonstrate an ability to collect newborn cells having synchronous growth. Additionally, it was observed that there was minimal contamination of cells of other ages. Based on the size distributions, the contamination with older cells is less than 5% for at least two generations, and probably considerably less for the first several hours of elution. The synchronous cells are minimally disturbed and thus can be used to investigate cell cycle processes during steady-state growth.

### Example 6

Suspensions of newborn cells of mouse and human origin were produced and analyzed. Human leukemic U937 cells (ATCC CRL 1593) were cultured in RPMI 1640 media supplemented with 2 g/l dextrose, 50 units/ml penicillin G, 50 mg/ml streptomycin sulfate and 10% fetal bovine serum in 75 cm² culture flasks. Human leukemic MOLT-4 cells (ATCC CRL 1582) were cultured in spinner flasks in L-15 medium supplemented with 2 gm/l glucose and 10% fetal bovine serum. Mouse L1210 cells were cultured as described in Example 1.

Each experiment was initiated with 2.2×10⁵ MOLT-4 cells/ml in 300 ml of L-15 medium Generation of newborn cell suspensions was achieved in a manner similar to that in Example 1 with some modifications. For U937 cells, all aspects of the experiments were performed in a 5% CO₂ atmosphere with 3.6×10⁵ U937 cells/ml in 175 ml of 1640 medium. The adhesives were concanavalin A for U937 and polylysine for MOLT-4. To reduce the number of cell aggregates attached to the surface, the cultures were filtered through a 12 μm-pore size Nucleopore polycarbonate filter immediately prior to attachment.

Newborn cell suspensions were collected from the effluent of immobilized populations and placed in culture flasks maintained in a 37° C. incubator. Samples were taken from these synchronous batch cultures at intervals [beginning at the end of collection of the effluent (0 h)] for determination of cell concentrations and cell size(s) using a Coulter Electronic Particle Counter/Channelyzer.

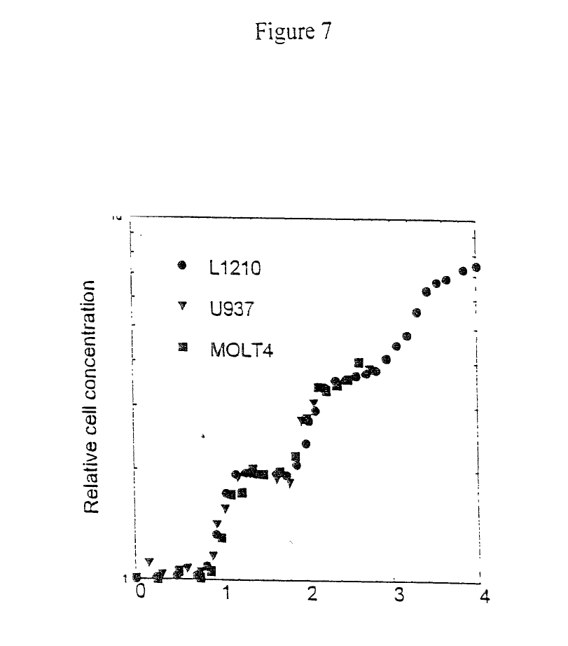

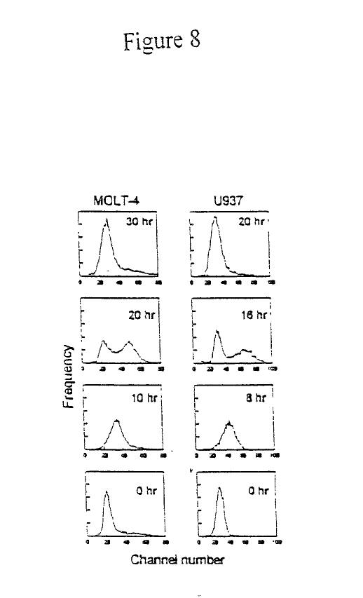

Consistent with the premise that the methodology is applicable to a wide variety of lympho-hematopoietic cells, the experiments with MOLT-4, U937 and L1210 exhibited clear synchronous growth and division (FIG. 7). Relative cell concentrations were plotted with respect to generations of growth for each cell line, with the generation times being approximately 9 h for L1210, 19 h for MOLT-4, and 21 h for U937. It is interesting to note that the shapes of the three synchrony curves were basically identical, leading to the unanticipated conclusion that the coefficients of variation in interdivision times of the mouse and human cell lines are indistinguishable. Importantly, the quality of synchrony was high, and the disturbance from steady-state so minimal, that all procedures for analysis of cell properties over multiple cycles are easily accomplished. For cell cycle work, it is essential to show that the results observed are the same over at least two cell cycles to demonstrate that the observations relate to true cell cycle events rather than an effect of the “treatment” used to produce the synchronous cells. The small newborn cells at time 0 gradually increased in size during synchronous growth until they reached double the newborn cell size and underwent synchronous division, shown at 20 h for MOLT-4 and 16 h for U937 (FIG. 8).

(A) MOLT-4 cells were grown in L-15 medium and attached to a polylysine-coated filter. Size distributions are shown at 0, 10, 20 and 30 h of synchronous growth of a sample collected between 4 and 4.3 h of elution. (B) U937 cells were grown in RPMI 1640 medium and attached to a conA-coated filter. Size distributions are shown at 0, 8, 16 and 20 h of synchronous growth of a sample collected between 3 and 4 h of elution.

### Example 7

The cells of Example 6 were analyzed for DNA and cyclin B1 content. Synchronous cell aliquots containing a minimum of 2×10⁵ cells were also fixed at the appropriate timepoints or cell densities with 10 ml/ml 1M sodium azide and centrifuged for 4 min at 2000 rpm. The supernatant was aspirated and the cells were resuspended in 1 ml of a wash buffer comprised of 3% heat-inactivated fetal bovine serum (FBS; Gibco) in Dulbecco's phosphate buffered saline.

For flow cytometric analysis of DNA content, the cells were collected by centrifugation, and the supernatant was removed via aspiration. The pellets were resuspended in 500 ml of PBS and vortexed, after which 500 ml of a solution containing 20 mg/ml propidium iodide and 50 mg/ml RNase A was added in low-light conditions. The cell suspensions containing propidium iodide were shielded from light and allowed to incubate at room temperature for a minimum of 20 min.

For flow analysis of cyclin B1 expression, samples were centrifuged for 4 min at 2000 rpm and the ethanol supernatant removed via aspiration. The pellets were then resuspended in 1 ml PBS and centrifuged once again. After aspiration of supernatant the pellets were resuspended in 100 ml PBS by gently vortexing. Ten ml of FITC-conjugated IgG monoclonal antibody (negative control) or FITC-conjugated cyclin B1 monoclonal antibody (PharMingen; La Jolla, Calif.) was added to the appropriate tube(s) and they were incubated for 30-40 min at 4° C. in the dark. Subsequently, 1 ml of PBS was added to each tube and the contents centrifuged. After removal of the supernatant, the pellets were resuspended in 0.5 ml of PBS and 0.5 ml of a solution containing 50 mg/ml propidium iodide and 50 mg/ml RNase A, and were incubated at room temperature for 20 min in the dark.

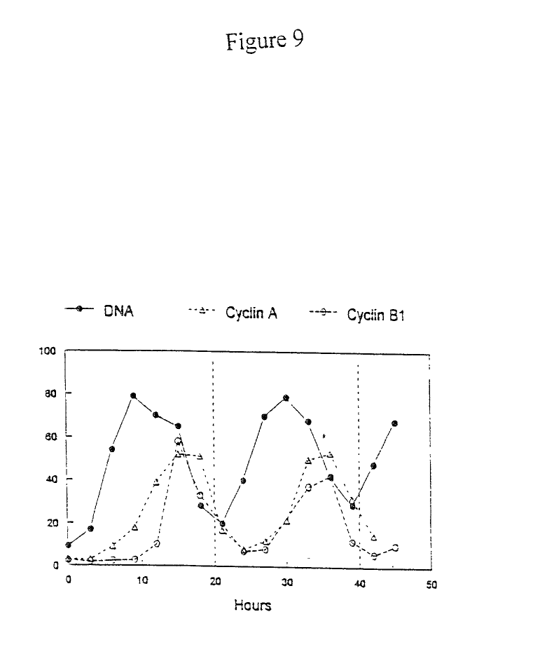

As an example of the determination of cell cycle gene expression, an experiment is shown in FIG. 9 in which synchronous human MOLT-4 cells were assayed for DNA replication with PE-conjugated anti-BrdU, the concentrations of cyclins A and B1 with FITC-conjugated cyclin A or B1 antibodies using a BD FACScan flow cytometer. Synchronous cell suspensions were assayed for DNA replication by labeling the cultures with BrdU for 30 min and analyzing incorporation with PE-conjugated anti-BrdU by FACS (●). The cellular contents of cyclins A (Δ) and B1 (∘) were determined with FITC-conjugated cyclin A and B1 antibodies by FACS. The interrupted lines indicate ends, on average, of the first and second cycles of synchronous growth. By analysis of at least two cycles in this manner, the timing of DNA replication, and the appearance and disappearance of the cyclins in the cycle are clearly evident.

### Example 8

Peripheral blood mononuclear cells were prepared from healthy volunteers by centrifugation with Sigma Histopaque-1077, and lymphocytes were activated with PHA and maintained growing in culture for approximately 7 days in L-15 culture medium containing interleukin-2. At this time, the cells were introduced into the device disclosed in Example 1, wherein the membranes were coated with either polylysine, conA or PHA and elution continued with interleukin-2-containing medium. In these studies, the experiments with conA proved most successful, as judged by the release of newborn cells.

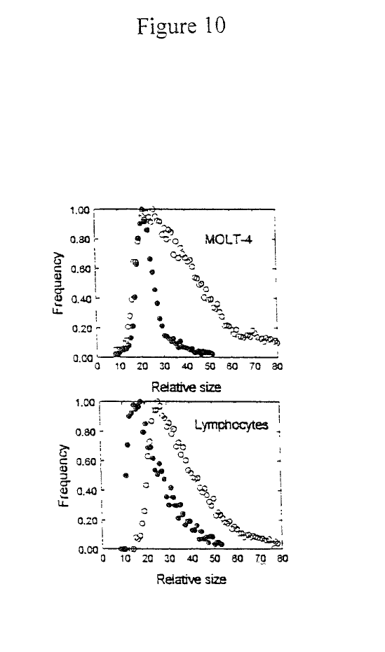

As shown in FIG. 10, the size distributions of the newborns were broader than those with the cell lines, but that is likely due to absence of a culture condition approaching steady-state growth prior to immobilization in the instrument. Relative cell sizes were determined, with a Coulter electronic particle counter with channelizer, in the effluent after approximately 0.3 generations of elution. However, the preliminary findings demonstrate that the culture system can be used as effectively with normal activated lymphocytes as with the established cells lines.

### Example 9

Immobilization by a pressure differential was developed in the present Example. The properties of cells held at the tips of glass micropipets (1 to 2 pin diameter) in growth medium, were examined, followed by subsequent growth and division by time-lapse video. The cells (bacteria, budding and fission yeast or mammalian cells) were drawn to the pipette tips with a syringe attached by fine tubing to the micropipets.

All cells grew and divided, and no difference was detected in the growth properties (e.g., interdivision times and cell sizes) of the immobilized cells vs. cells in suspension. In all the cultures examined, the originally attached cell remained attached. From these findings, the preferred use for the cell culture technology is to hold the cells to a large number of holes in ordered arrays in a solid surface.

Ordered arrays were selected because random arrays were sometimes so close together that adjacent cells either interacted or the two cells could not cover both holes. Thus, discs manufactured by micro lithography, containing ordered arrays of 1.5 μm-diameter holes spaced 13 μm apart in 6 μm thick electroformed nickel were obtained and tested. The pore sizes and spacings are functions of the cells being used, it was determined that discs with 2 μm diameter holes spaced 10-20 μm apart are appropriate for mammalian cells, and 1 μm diameter holes spaced 3-5 μm apart for bacteria and lower eukaryotes. The discs were clamped in a standard membrane filter holder, and inverted into growing populations at 10⁶-10⁸ cells/ml. Cells are drawn to the holes in the discs, and the discs are held in place in the top section by the differential pressure. Once each hole was plugged with a cell, no further movement of medium was detectable. The upper section was then screwed or clamped to the lower section containing the inlet and outlet ports for medium flow. For studies on the newborn cells shed from the surface, medium flows over the cells continuously, and samples of the effluent can be collected whenever desired. For experiments on the cells on the surface, the cells are simply removed from the filter disc and examined.

The sizes of the attachment surfaces, and as a consequence the number of cells and the medium utilization, can span a wide range. For use in monitoring cell growth properties, the attachment surface can be about 1 cm in diameter, with a narrow (ca. 100 μm) bottom section, and contain cell numbers ranging from 10⁷ bacteria to 10⁵ large mammalian cells. It has been found that medium flow rate can be successfully set at 0.002 ml/min/cm² for all cells, that is, about 3 ml/day. As a consequence, medium utilization is very low with this system. The medium can be reduced at least 3-fold more and still maintain the cells in steady state growth. Thus, the small units will consume not more than 1 liter/year, and preferably, considerably less. This is due to the principle upon which this continuous culture system is based, and is not achievable in a batch culture device. Furthermore, shear stress on the cells is very low since there is no stirring or periodic medium replacement.

Large units for research and product development to produce larger quantities of cells of uniform age, with a surface area of 100 cm², will contain about 5×10⁷ mammalian cells, and medium flow rates of about 300 ml/day. This produces a quantity of newborn cells sufficient for most biochemical studies.

The initial tests were performed with L1210 mouse lymphocytic leukemia cells because they grow well in suspension and do not bind to, or spread on, the nickel discs. Actively growing cells were drawn to, and held on, the discs with a very small differential pressure, and growth of the attached cells, held on the bottom surface, was monitored by time-lapse video using bright field reflected light in an inverted microscope. FIG. 11a shows an example of the array of cells held to discs taken from a videotape of the process. As seen with reflected light, the tiny dark spots in the centers of the circles are the holes arranged in the array seen through the cells (larger spherical objects). To aid visualization, FIG. 11b shows the same field slightly out-of-focus. There are 6 “plugged” holes without cells in this array of 180, e. g., all holes have cells attached in column 6 except for row 8. It has been found that the cells can be held indefinitely on the surface as shown, and will grow and divide (e.g., the cell in column 10, row 7) while fixed in place. The optimal pressure differential can vary depending on cell type, but it is preferably very slight, certainly less than the 0.5 psi used to hold cells during micromanipulation, and appears to have no measurable effect on cell growth. In fact, submersing the nickel disc several millimeters below the surface was sufficient to draw, capture and hold the cells in place. Since the cells grew while held on the holes, the differential pressure could be eliminated entirely and the cells remained. The formation of cell surface components sealed the cells to the holes so that positive pressure was actually needed to remove the attached cells after two or three generations. Since we already know that such molecular adhesions are never permanent, long-term growth in this manner still requires a differential pressure, but its magnitude can be decreased even more once the seal forms.

### Example 10

To achieve attachment of animal cells, it was originally believed that the cells had to grow and divide while attached to tiny adhesive sites distributed over a non-adhesive surface. The sites had to be smaller than the diameter of a newborn cell so each time a cell divided there was usually room for only one new daughter cell to remain attached while the other was released. To produce the non-adherent surface, either a glass plate or the bottom of a 25-ml polystyrene culture flask was coated with a 5% solution of poly-2-hydroxyethyl methacrylate (polyHEMA) in absolute methanol. The adhesive sites consisted of either 4.8 mg-diameter adhesive beads, or holes, through the polyHEMA generated by placing a glass plate with a mask with small holes over the coating in a glow discharge chamber. In the experiments, Chinese hamster ovary cells (CHO-K1, ATCC CCL 61) were added to the prepared surfaces, and the cells attached only to the beads or holes.

The findings in these experiments led to two important discoveries. First, it was found that the attached cells divided in planes that were randomly distributed with respect to the plane of attachment, that is not perpendicular to the plane as is the case with cells spread on surfaces. It was also found that successive divisions were in orthogonal planes. This finding led to the unexpected conclusion that newborn cell suspensions could be obtained by growing non-adherent cells, such as hematopoietic cells, on uniformly adhesive surfaces since the axis of division would be away from the binding surface in most instances so that one of the two daughters could release from the surface. Secondly, it was observed that cells did not remain permanently fixed to the prepared surfaces through the chemical/affinity attachment. Thus, although short-term production of newborn cell suspensions is practical, long-term growth requires a permanent physical attachment, which led to the development of the additional features of the current invention. Based on these preliminary findings, newborn hematopoietic cells are produced according to the current invention by binding a growing population of cells to a uniformly adhesive surface and flushing the surface continuously with culture medium. When the immobilized cells divide, the new daughter possessing the attachment site on its surface remains bound while the other new daughter is released. When the entire surface is adherent, both daughters may remain attached in a proportion of the divisions, about 10%. Most of the divisions result in release of one newborn because, as described above, the division planes are randomly positioned with respect to the plane of the attachment surface, and thus rarely perpendicular to the surface.

Thus, there has been shown and described a method for producing synchronized cell cultures, which fulfills all the objects and advantages sought therefore. It is apparent to those skilled in the art, however, that many changes, variations, modifications, and other uses and applications for the production of synchronized cell cultures are possible, and also such changes, variations, modifications, and other uses and applications which do not depart from the spirit and scope of the invention are deemed to be covered by the invention, which is limited only by the claims which follow.

### REFERENCES

Cooper, S. (1998) “Mammalian cells are not synchronized in G1-phase by starvation or inhibition: considerations of the fundamental concept of G1-phase synchronization,” Cell Prolif. 31:9-16.

Gong, J., Traganos, F., and Darzynkiewicz, Z., (1995) “Growth imbalance and altered expression of cyclin B1, A, E, and D3 in MOLT-4 cells synchronized in the cell cycle by inhibitors of DNA replication,” Cell Growth Differ, 6:1485-1493.

Grdina, D. J., Meistrich, M. L., Meyn, R. E., Johnson, T. S. and White, R. A., (1987) “Techniques in Cell Cycle Analysis, Gray,” J. W. and Darzynkiewicz, Z., eds. Humana Press, pp. 367-402.

Grdina, D. J. et al [1987] Humana Press 367-402, Merrill, G. F. [1980] Methods in Cell Biol. 57:229-249, Zickert, P. et al. Exp. Cell Res. 207:115-121).

Helmstetter, C. E. (1969) “Methods for studying the microbial division cycle,” In: J. R. Norris and D. W. Ribbons (ed.), Methods in microbiology, vol. 1. Academic Press, New York p. 327-363).

Helmstetter, C. E., Eenhuis, C., Theisen, P., Grimwade. J. and Leonard, A. C., (1992) “Improved bacterial baby machine: Application to Escherichia coli K12 with increased cell yield,” J. Bacteriol., 174:3445-3449.

Helmstetter, C. E. (1991) “Description of a baby machine for Saccharomyces cerevisiae,” New Biol., 3:1089-1096.

Helmstetter, C. E. (1995) “Preferential release of one daughter cell during division of immobilized CHO cells,” Biotech. Bioeng., 45:373-377.

Helmstetter, C. E. (1997) “Gravity and the orientation of cell division,” Proc. Natl. Acad. Sci. USA, 94:10195-10198.

Kauffman, M. G., Noga, S. J., Kelly, T. J., and Donnenberg, A. D. (1990) “Isolation of cell cycle fractions by counterflow centrifugal elutriation,” Anal. Biochem., 191:41-46.

Knehr, M., Poppe, M., Enulescu, M., Eickelbaum, W., Stoehr, M., Schroeter, D., and Paweletz, N. (1995) “A critical appraisal of synchronization methods applied to achieve maximal enrichment of HeLa cells in specific cell cycle phases,” Exp. Cell Res., 217:546-553.

Lloyd, D., Poole, R. K. and Edwards, S. W. (1982) “The Cell Division Cycle,” Academic Press.

Merrill, G. F. (1980) “Cell synchronization,” Methods in Cell Biol., 57:229-249.

Mitchison, J. M. (1971) “The Biology of the Cell Cycle,” Cambridge Univ. Press.

Terasima, T. and Tornach, L. J. (1963) “Growth and nucleic acid synthesis in synchronously dividing populations of HeLa cells,” Exp. Cell Res., 30:344-362.

Thilly, W. G. (1976) “Maintenance of perpetual synchrony in HeLa S3 cultures: theoretical and empirical approaches,” In: Methods in Cell Biology Prescott, D. M., 14:273-285.

Tobey, R. A. (1973) “Production and characterization of mammalian cells reversibly arrested in G1 by growth in isoleucine-deficient medium,” Methods in Cell Biology. ed. by Prescott, D. M., 6:67-112.

Urbani, L., Sherwood, S. W., and Schimke, R. T. (1995) “Dissociation of nuclear and cytoplasmic cell cycle progression by drugs employed in cell synchronization,” Exp. Cell Res., 219:159-168.

Zickert, P., Wejde, J., Skog, S., Zetterberg, A. And Larsson, O. (1993) “Growth-regulatory properties of G, cells synchronized by centrifugal elutriation,” Exp. Cell Res., 207:115-121.

What is claimed is:

1. A method for obtaining a culture of cells that are minimally perturbed and in a steady state of growth, comprising:
(a) obtaining a culture of cells that are growing exponentially;
(b) adhering said cells of said culture to a surface by contacting said culture with said surface;
(c) continuously flushing said adhered cells with an amount of culture media; and,
(d) collecting said media effluent containing newborn daughter cells derived from said adhered cells, in order to obtain said culture of cells.

2. The method of claim 1, wherein said cells are mammalian cells.

3. The method of claim 2, wherein said mammalian cells are selected from the group consisting of hematopoietic cells.

4. The method of claim 1, wherein said cells are adhered to the surface as a result of an adhesion composition being applied to the surface, whereby said cells are attached to said surface.

5. The method of claim 4, wherein said composition is selected from the group consisting of charged molecules, antibodies, ligands, compounds that will form covalent bounds with surface molecules, lectins, agglutinins, and combinations thereof.

6. The method of claim 1, wherein said surface is selected from the group consisting of a filter membrane and flat surface.

7. A culture of cells, wherein said cells are minimally perturbed, of a uniform size and age distribution reflective of newborn cells in an exponentially growing culture, and of a synchronous progression through cell mitotic cycle, including cycle-specific replication, transcription, and translation.

8. The culture of claim 7, wherein said cells are selected from the group consisting of hematopoietic cells.

9. The culture of claim 7, wherein said culture has at least 10³ newborn cells/ml.

10. The culture of claim 7, wherein said culture is comprised of cells in S phase of cell division cycle.

11. The culture of claim 7, wherein said culture is comprised of cells in G₁ phase of cell division cycle.

12. The culture of claim 7, wherein said culture is comprised of cells in G₂ phase of cell division cycle.

13. The culture of claim 7, wherein said culture is comprised of cells in M phase of cell division cycle.

14. The culture of claim 7, wherein said culture is generated at 10³ cells/min/cm² adherent surface area.

15. The culture of claim 7, wherein said culture is generated at 5×10⁵ cells/generatation/cm² adherent surface area.

16. The culture of claim 7, wherein said culture has uniform size and age distribution reflective of newborn cells in an exponentially growing culture.

17. The culture of claim 7, wherein said culture produces at least 200 cells/minute.

18. A suspension of newborn cells from growth-activated normal mammalian cells for research on a normal cell cycle.

19. The cell suspension of claim 18 comprising normal cells from mammals of varying ages for research on cell cycle aspects of cellular senescence.

20. The cell suspension of claim 18, comprising cells cultured for extended periods in vitro for research on cell cycle aspects of cellular senescence.

21. The cell suspension of claim 18, comprising pluripotent hematopoietic stem cells.

22. A method for obtaining a culture of cells that are minimally perturbed and in a steady state of growth, comprising:
(a) obtaining a culture of cells selected from the group consisting of hematopoietic cells that are growing exponentially;
(b) adhering said cells of said culture to a surface by contacting said culture with said surface, whereby a composition selected from the group consisting of charged molecules, antibodies, ligands, compounds that will form covalent bounds with surface molecules, lectins, agglutinins, and combinations thereof,
(c) continuously flushing said adhered cells with an amount of culture media at a media flow rate equal to about 300 ml per day; and,
(d) collecting said media effluent containing newborn daughter cells derived from said adhered cells, in order to obtain said culture of cells.

23. A culture of parent cells for producing daughter cells that are minimally perturbed, of a uniform size, and of a synchronous progression through DNA replication, wherein said parent cells form an immortal cell line, and are of a mammalian origin.

24. The culture of claim 23, wherein said composition is adhered to a surface with a composition selected from the group consisting of charged molecules, antibodies, ligands, compounds that will form covalent bounds with surface molecules, lectins, agglutinins, and combinations thereof.

25. A culture of cells, wherein said cells are minimally perturbed, of a uniform size and age distribution reflective of newborn cells in an exponentially growing culture, and of a synchronous progression through cell mitotic cycle, including cycle-specific replication, transcription, and translation, wherein said cells are selected from the group consisting of hematopoietic cells.
